(19) United States
(12) Patent Application Publication
(10) Pub. No.: US 2006/0173332 A1
(43) Pub. Date: Aug. 3, 2006

(54) Detection of small-size defects in medical ultrasonic imaging

(76) Inventors: Claire Prada, Paris (FR); Claude Chohen-Bacrie, New York, NY (US); Mathias Fink, Meudon (FR); Robert Randall Entrekin, Kirkland, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(21) Appl. No.: 10/538,155

(22) PCT Filed: Dec. 17, 2003
(86) PCT No.: PCT/IB03/06033

§ 371 Date: Mar. 10, 2006

(30) Foreign Application Priority Data
Dec. 30, 2002 (FR) 02 16861

(51) Int. Cl. A61B 8/06

(52) U.S. Cl. 600/447

(57) ABSTRACT

The invention relates to an ultrasonic medical imaging apparatus intended for analyzing an organic medium potentially including defects within a noisy structure. Ultrasound signals focused at a given depth according to M distinct successive excitations arc emitted and an image formation module makes it possible to obtain an image of said depth after reception of responses from the medium. The invention is such that the apparatus includes a module for using said responses for constructing a rectangular response matrix of dimension N*M, a coefficient Knm of which represents the response from the medium received by the transducer n following an excitation m, decomposing said response matrix into singular values, using the singular vectors corresponding to said singular values for locating singular zones corresponding to defects in the medium. Application: Ultrasonic apparatus particularly in the medical field.

The invention relates to a method of analyzing an organic medium potentially including defects within a noisy structure, said medium being excited by ultrasonic signals emitted by a set of transducers. The invention in particular relates to medical imaging and the advanced functions which can be implemented in ultrasonic imaging apparatus. The invention concerns in particular imaging of the breast and the detection of microcalcifications in the breast.

Such a method is known from the article “Ultrasonic Nondestructive Testing of Scattering Media using the Decomposition of the Time-Reversal Operator”, published in IEEE Transactions on Ultrasonics, Ferroelectrics and Frequency Control, Vol. 49, N^{o} 8, August 2002, by E. Kerbrat, C. Prada, D. Cassereau and M. Fink. The known method proposes to study the medium using the decomposition of the time-reversal operator. According to the method proposed, a first transducer in an array of transducers is excited by a short excitation and the signals resulting from the response of the medium are received on all the transducers in said array. This operation is repeated for each of the transducers with the same excitation. A square transfer matrix K is then obtained by producing a Fourier transform of the responses of the medium. The time-reversal operator is then defined by K*K and can be diagonalized. The number of significantly non-zero proper values is equal to the number of defects detected by said method. Said defects are then located by means of a calculation of the proper vectors.

The method proposed in this document has the drawback of requiring many excitations particular to the method. The particular character of these excitations makes it possible to take account of only some of the information present in the medium. This method must therefore be used in parallel with and independently of other insonifications of the medium malting it possible to have access to other information, for example in order to obtain an image of the medium. In addition, these particular excitations cannot be carried out by a common ultrasonic imaging apparatus and the use of a specific apparatus is therefore obligatory. This is a problem in the field of medical imaging, this field requiring easy and rapid acquisition of data for applications requiring results that are rapid or even in real time. In addition, according to the method disclosed here, little energy is transmitted to the medium and this results in a limited propagation of the ultrasound. This limited propagation does not make it possible to form correct ultrasonic images.

One object of the invention is to provide a method of analyzing a medium potentially including defects within a noisy structure, said medium being excited by ultrasonic signals not having the drawbacks of the method of the prior art.

The object of the invention is achieved by means of an analysis method in accordance with the introductory paragraph such that the ultrasonic signals are focused at a given depth according to M distinct successive excitations in order to obtain an image of said depth after reception of the responses from the medium, such that it also includes the steps of:
constructing a rectangular response matrix of dimension N*M, a coefficient Kₙₘ of which represents the response of the medium received by the transducer n following an excitation m,
decomposition of said response matrix into singular values,
use of the singular vectors corresponding to said singular values in order to locate singular zones corresponding to defects in the medium.

According to the invention, the medium is excited according to focusings conventionally used in ultrasonic imaging, for example centered on successive transducers or on successive geometric fractions of the transducers in an array of transducers.

The invention uses the echographic responses of the medium received individually on each of the transducers in order to locate defects within the medium. The invention therefore does not require any particular excitations of the medium and therefore makes it possible to effect a single data acquisition. In addition, the invention can be implemented in an ultrasonic imaging apparatus with minor modifications in order to obtain an appreciable improvement in the detection and location of reflectors which are the origin of the singular zones by localized modification of the reflection properties.

In a first embodiment, a matrix of responses Kₙₘ is obtained for a plurality of frequencies. It will in fact be noted hereinafter that the various singular values do not appear with the same intensity for all frequencies. It may thus be advantageous to construct several response matrices, each for one frequency from a plurality of frequencies.

In an advantageous embodiment, M successive excitations are effected for a plurality of depths of said medium. This is generally carried out in order to acquire an image and advantageously used for constructing response matrices at various depths and in order to have detection of singular zones on an extended zone of the medium.

In a preferred embodiment of the invention, the step of using the proper vectors for locating a singular zone results in the formation of a binary image of the medium: the value 1 being allocated to the zones for which the presence of a coherent reflector is detected, the value 0 being allocated elsewhere (for example in the noisy zones). The purpose of this embodiment is to allow display of the information given by the method according to the invention and in particular to allow exploitation of this information in order, for example, to adapt the insonification of the medium, to process the zones differently where a defect is detected, etc.

The invention makes it possible to introduce into any ultrasonic apparatus means of detecting a coherent reflector within a medium from which the noisy data results. Thus the invention also relates to an apparatus intended to analyze a medium potentially including defects within a noisy structure, said apparatus including a set of transducers for emitting ultrasonic signals focused at a given depth according to M distinct successive excitations, an image formation module in order to obtain an image of said depth after reception of the responses from the medium, such that it includes a module for exploiting said responses in order:
to construct a rectangular response matrix of dimension N*M, a coefficient Kₙₘ of which represents the response of the medium received by the transducer n following an excitation m,
to decompose said response matrix into singular values,
to use the singular vectors corresponding to said singular values in order to locate singular zones corresponding to defects in the medium.

Thus the invention finds an advantageous application in the field of medical imaging and particularly in ultrasonic imaging for which the images obtained are conventionally noisy and the coherent reflection modifications difficult to detect. An apparatus according to the invention is thus conventionally a medical imaging station.

By making it possible to locate coherent reflectors which are not visible on a combined image, the invention also contributes to the refinement of the imaging results and may allow more precise and more accurate diagnosis in the particular case of medical imaging.

The invention also relates to a signal exploitation module able to be inserted in an imaging apparatus including a set of transducers for emitting ultrasonic signals focused at a given depth according to M distinct successive excitations, an image formation module for obtaining an image of said depth after reception of the responses from the medium, said signal exploitation module being intended to exploit said responses from the medium by:
constructing a rectangular response matrix of dimension N*M, a coefficient Kₙₘ of which represents the response from the medium received by the transducer n following an excitation m,
decomposing said response matrix into singular values,
using the singular vectors corresponding to said singular values in order to locate singular zones corresponding to defects in the medium.

The invention will be further described with reference to examples of embodiments shown in the drawings to which, however, the invention is not restricted.

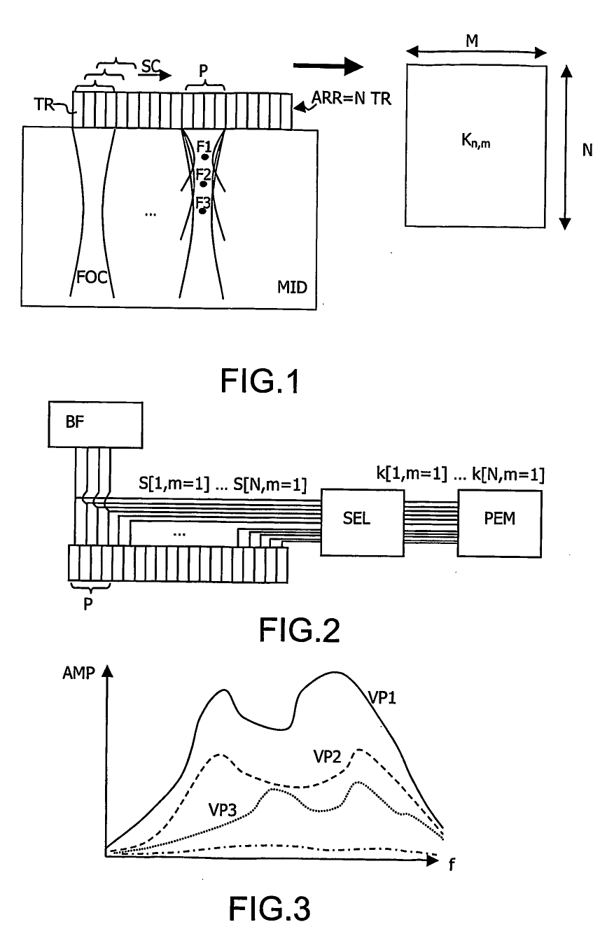

FIG. 1 is a diagram explaining the reception according to the invention of the echographic signals coming from a medium and the formation of the rectangular response matrix,

FIG. 2 is a partial functional diagram of an ultrasonic imaging apparatus implementing the invention,

FIG. 3 is a graph of the singular values obtained according to the first embodiment of the invention,

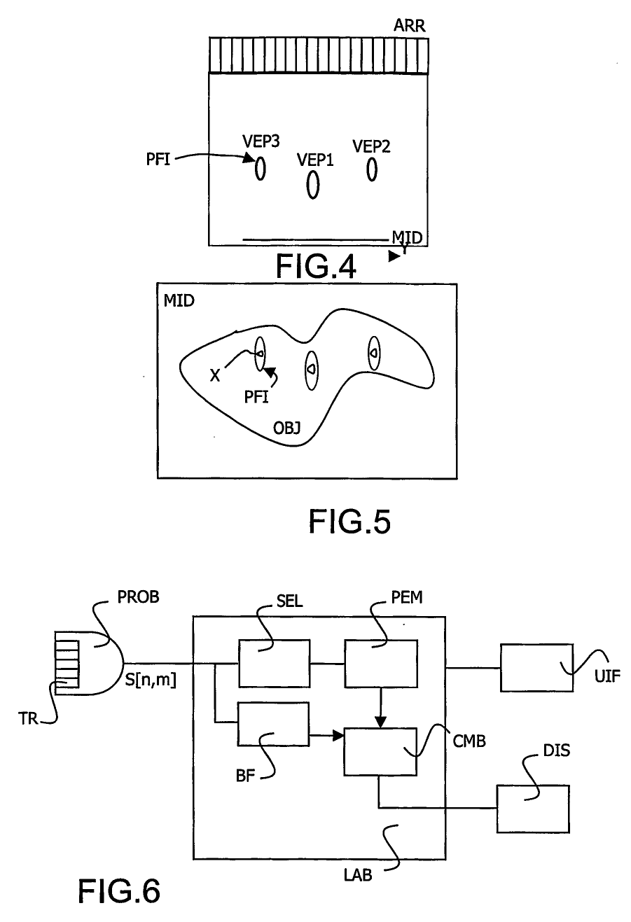

FIG. 4 illustrates the location of a singular zone according to the invention,

FIG. 5 illustrates an application of the invention to an ultrasonic image,

FIG. 6 is a general functional diagram of an apparatus for analyzing a medium according to the invention.

The following remarks relate to the reference signals. Similar entities are designated by identical letters in all the Figures. Several similar entities may appear in a single Figure. In this case, a digit or a suffix is added to the letter reference in order to distinguish similar entities. The digit or suffix may be omitted for reasons of convenience. This applies to the description and to the claims.

The description which follows is presented so as to enable a person skilled in the art to implement and make use of the invention. Various alternatives to the preferred embodiment will be obvious to a person skilled in the art and the generic principles of the invention disclosed here may be applied to other embodiments. Thus the present invention is not deemed to be limited to the embodiment described, but rather to have the widest scope in accordance with the principles and characteristics described below.

FIG. 1 depicts a diagram explaining the reception of echographic ultrasonic signals coming from a medium MID according to the requirements of the invention. The medium MID is excited by focused ultrasonic waves FOC. The focusing is carried out on P transducers TR in an array of transducers ARR centered on the geometric middle of these P transducers. According to FIG. 1, P=4. The focusing techniques also make it possible to center the wave at any point on the array of transducers. According to the invention, the echographic signals returned by the medium MID are then recorded on each of the N transducers AR in the array ARR of transducers TR. According to the ultrasonic acquisition methods conventionally used in medical imaging, the excitation is then repeated according to the same focusing on P transducers but offset with respect to the previous one in a scanning direction indicated for example in FIG. 1 by the arrow SC. Thus, according to a conventional scanning, M acquisitions are made. The acquisition number M may vary and is generally different from the number N of transducers TR in the array AR of transducers TR. In addition, the conventional acquisitions are made generally and according to an advantageous embodiment of the invention for various focusing depths represented by the points F1, F2, F3. Each acquisition at a given depth gives particular information at said depth. However, these excitations do not make it possible to access the inter-element responses to a pulse which are essential to the implementation of the proposed method of the prior art.

FIG. 2 is a partial functional diagram of an ultrasonic imaging apparatus implementing the invention. This functional diagram shows more particularly an acquisition made according to the invention using a first excitation m=1 made by a broad frequency spectrum wave focused on the first P transducers in the array of transducers. For example, the spectrum is centered on a frequency of 3 to 5 MHz and possesses a bandwidth of 40% of the total bandwidth. The echographic signals S[n=1, m=1] . . . S[n=N, m=1] are received independently by each of the transducers n, ε [1, N] following the excitation m=1 and are transmitted to beam formation means BF so as then to generate an ultrasonic image according to the means known to persons skilled in the art. This ultrasonic image generally makes it possible, referring to FIG. 5, to see the medium MID and an object OBJ included in this medium MID but does not in general make it possible to distinguish a defect X of small size in the generally noisy image, notably presenting a speckle noise. This object may for example be an organ and the small defect be a reflector due to the presence of an ailing area. Thus, with the breast, the small defect may be a microcalcification. According to the invention, the echographic signals S[1, m=1] . . . S[N, m=1] (also denoted Sₙ₁) received independently by each of the transducers n in the entire array of transducers are also transmitted to a selector SEL which selects a time part of the signal received at each transducer. This time part generally corresponds to the signals received coming from the vicinity of a focusing point F(1, 2 or 3) as presented in FIG. 1. The magnitude of this vicinity depends on the compromise which the user wishes to have between detection at a great depth and the precision of this detection. A scanning of m excitations is then carried out along the array of transducers according to the techniques conventionally used in medical ultrasonic imaging. Time parts of the signals received S[1, m] . . . S[N, m] are selected for each of the excitations m of a scanning of the medium of M excitations, m thus being included in [1, M]. These signal time parts are denoted kₙₘ or k[n, m] and are time functions. These signal parts k[n, m] are then transmitted to a module PEM for the particular exploitation of the signals. For each excitation m of the scanning, this module PEM stores in memory the part kₙₘ of the response function Sₙₘ received by a transducer n from the medium MID corresponding to a certain depth interval. The Fourier transforms of the signals kₙₘ(t) give the matrix K=(Kₙₘ(ω))_{1≦n≦N;1≦m≦M} which is referred to as the response matrix. Thus a matrix of coefficients Kₙₘ is obtained each representing the response of the medium for a given excitation frequency received by the element n following a focused excitation m of the medium. This matrix is rectangular and can be calculated for each frequency of the spectrum, generally according to a making discrete thereof. It is possible to acquire only one matrix for a single frequency but the result may be less precise. The frequencies chosen may also be selected in order to comply with the constraints of resolution and attenuation in the medium. Such frequencies are in fact dictated, according to the invention, by the acquisition of the conventional ultrasonic image of the medium.

The module PEM next calculates the decomposition into singular values of the response matrix K. Effectively, this decomposition is in particular used for the resolution of a singular system and a rectangular matrix of dimension NM with real or complex coefficients may be decomposed in the form K=UDV with U the unitary matrix of dimension NN and V the unitary matrix of dimension MM and D a diagonal matrix of dimension NM. The diagonal elements of the matrix D of dimension NM are simply the square roots of the singular values of the matrix K^{#}K where K^{#} is the conjugate of the transpose.

The singular vectors of the matrix K^{#}K are the columns of U.

In a first embodiment, the module PEM calculates a plurality of response matrices for a plurality of frequencies. In this case a graph representing the amplitude AMP of the singular values VP according to the frequency f as presented in FIG. 3 is obtained. The singular values VP1, VP2, VP3 do not all appear with the same relative intensities for the same frequency values. A defect in a medium is marked by a local change in reflection of the signal and therefore can be considered and defined by the reflector term. The correspondence between the presence of a reflector and the presence of a non-zero singular value was studied, for the simple method of diagonalization of the time-reversal operator, in the document “Eigenmodes of the Time-Reversal Operator: A Solution to Selective Focusing in Multiple-Target Media”, C. Prada, M. Fink, Wave Motion 20/1994, pp. 151-163. It is observed that the invention makes it possible also to obtain this correspondence. Thus, according to the invention, a non-zero singular value of the rectangular matrix constructed in the frequency domain studied reveals the presence of a reflector. The correspondence is therefore a singular value=a reflector and the largest singular value corresponds to the largest reflector.

According to the first embodiment, a plurality of matrices is constructed for various frequencies and an inverse Fourier transform of the proper vectors, that is to say the columns of the matrix U, can then be calculated. This makes it possible to obtain the singular time vectors which correspond to the singular frequency vectors. This makes it possible to propagate simply in return the singular time vectors in the medium so as to determine the pressure fields whose maxima correspond to the defects in the medium. This return propagation of the singular vectors in phase and amplitude uses for example the reception focusing techniques and is generally done by digital means which simulate the acoustic field within the medium. In practice a software performs this function of notional time wave propagation. For example, in FIG. 4, this first technique of propagation of a singular time vector results in coloring in a certain way the high pressure zones PFI. An image with color levels can also be obtained.

Software can also be proposed for reconstructing a propagation matrix for each depth of the medium thus made discrete. A matrix for passage from the plane of the sensor to the plane of a given depth is then obtained, making it possible to locate a reflector on the dimension Y. This reconstruction for various depths can, using the advantageous embodiment, be the result of an insonification of the medium according to waves focused at various depths and in a way which is made discrete. For example, in FIG. 1, three focusings of depth F1, F2, F3 are effected and a propagation matrix is constructed for these three depths. The two techniques of locating the singular zones proposed above make it possible to isolate one zone and in practice to display a pressure field PFI on a conventional ultrasonic image of a medium MID including an object OBJ as presented in FIG. 5. A marking is therefore carried out on an ultrasonic image of the zone studied in order to locate the defect or defects. For example, by virtue of the invention, a microcalcification is detectable in the breast whereas it may not emerge from the noise (“speckle”) on a conventional ultrasonic image.

The invention can be implemented for all types of medical imaging with ultrasonic acquisition. It is possible to use, according to the invention, the responses given by an organic medium after excitations according to various types of focusing used in ultrasonic imaging. The line density (that is to say the geometric interval between two successive excitations) may also be adapted independently of the invention so as to make the results given by the invention more precise laterally. It is thus possible to have a number M greater than N. In this case the system to be resolved is degenerated.

The invention can also be used to form an adaptable insonification beam: the singular vectors make it possible to send a strong pressure field on a tricky area and consequently make it possible to have more precise information on this area.

FIG. 6 depicts schematically an apparatus in which a method according to the invention is implemented. The invention can be implemented non-removably or in a modular form, a reflector detection module being added to a conventional ultrasonic apparatus. This detection module receives the echographic signals independently of the transducers of the ultrasonic apparatus and includes, for example, a selector SEL and an exploitation module PEM as described above. In FIG. 6, an apparatus in which the invention is permanently is depicted.

This apparatus includes a probe PROB including reception elements TR, said probe being connected by conventional means to a data processing apparatus LAB. In addition to a module BF for forming a return beam and an image according to the known techniques of ultrasonic imaging, any data processing apparatus LAB includes a selector SEL and an operating module PEM as described previously. The apparatus LAB is connected to a display module DIS which displays, by means of conventional display functions, in addition to the images conventionally obtained by an ultrasonic apparatus, the images which can be constructed from information obtained by virtue of the module PEM. A combination module CMB combines for example the data obtained by beam formation means BF and those obtained by the operating module PEM. Next the combination module CMB is connected to the display module DIS. Thus an image as presented in FIG. 5 and locating the singular zones can be obtained according to the invention. Any means of graphical representation of the singular zones (binary image, surrounding the zone etc) can be used indifferently for application of the invention. A user interface UIF is advantageously connected to the apparatus LAB for controlling this apparatus and parameterizing it: for example, a detection threshold value can be modified by the user as well as the depth incrementation value which can determine the precision of the location/detection of a reflector, the object of the invention.

The invention makes it possible to obtain precise location of any reflecting defects within a homogeneous medium for which noisy signals (“speckle”) are obtained, signals within which it is generally difficult to detect such defects with the known means. In one of its applications, the invention advantageously concerns compound imaging consisting of insonifying a medium in different directions and combining the results so as to obtain a more complete and less noisy image.

The modules presented previously for fulfilling the functions presented in the steps of the method according to the invention can be integrated as an additional application in a conventional ultrasonic apparatus or be used in an independent apparatus intended to be connected to a conventional ultrasonic apparatus for fulfilling the functions according to the invention. There exist many ways of implementing the functions presented in the steps of the method according to the invention by software and/or hardware means accessible to persons skilled in the art. This is why the Figures are schematic. Thus, although the Figures show various functions performed by various units, this does not exclude a single software and/or hardware means making it possible to fulfill several functions. Nor does this exclude a combination of software and/or hardware means making it possible to fulfill a function. Although this invention has been described in accordance with the embodiments presented, a person skilled in the art will immediately recognize that there exist variants to the embodiments presented and that these variants remain within the spirit and scope of the present invention. Thus many modifications can be achieved by a person skilled in the art without for all that being excluded from the spirit and scope defined by the following claims.

1. A method of analyzing an organic medium potentially including defects within a noisy structure, such that said medium is excited by ultrasonic signals emitted by a set of N transducers and focused at a given depth at M distinct successive excitations in order to obtain an image of said depth after reception of the responses from the medium, such that it also includes the steps of:
constructing a rectangular response matrix of dimension N*M, a coefficient Kₙₘ of which represents the response of the medium received by the transducer n following an excitation m,
decomposition of said response matrix into singular values,
use of the singular vectors corresponding to said singular values in order to locate singular zones corresponding to defects in the medium.

2. A method as claimed in claim 1, according to which a response matrix Kₙₘ is obtained for a plurality of frequencies.

3. A method as claimed in claim 1, according to which M successive excitations are carried out for a plurality of depths of said medium.

4. An ultrasonic medical imaging apparatus intended for analyzing a medium potentially including defects within a noisy structure, said apparatus including a set of transducers for emitting ultrasonic signals focused at a given depth according to M distinct successive excitations, an image formation module in order to obtain an image of said depth after reception of the responses from the medium, such that it includes a module for exploiting said responses in order:
to construct a rectangular response matrix of dimension N*M, a coefficient Kₙₘ of which represents the response of the medium received by the transducer n following an excitation m,
to decompose said response matrix into singular values,
to use the singular vectors corresponding to said singular values in order to locate singular zones corresponding to defects in the medium.

5. An apparatus as claimed in claim 4, such that a response matrix Kₙₘ is constructed for a plurality of frequencies.

6. An apparatus as claimed in claim 5, according to which M successive excitations are carried out for a plurality of depths of said organic medium.

7. (canceled)

8. A method as claimed in claim 2, according to which M successive excitations are carried out for a plurality of depths of said medium.

9. An apparatus as claimed in claim 4, according to which M successive excitations are carried out for a plurality of depths of said organic medium.
